SEQUENCE LISTING (2 sequences)

SEQ ID NO: 1 - DNA, 20 bp, Artificial Sequence
  primer
gtcttcaccaccatggagaa

SEQ ID NO: 2 - DNA, 21 bp, Artificial Sequence
  primer
ttcaccaccttcttgatgtca

(19) United States
(12) Patent Application Publication
(10) Pub. No.: US 2015/0225791 A1
(43) Pub. Date: Aug. 13, 2015

(54) BIOMARKER FOR VASCULAR ENDOTHELIAL FUNCTION

(71) Applicant: UNIVERSITY COURT OF THE UNIVERSITY OF DUNDEE, Dundee (GB)

(72) Inventors: Aleksandar Jovanovic, Dundee (GB); Faisel Khan, Dundee (GB)

(21) Appl. No.: 14/418,943

(22) PCT Filed: Jul. 31, 2013
(86) PCT No.: PCT/GB2013/052053

§ 371 Date: Jan. 31, 2015

(30) Foreign Application Priority Data
Aug. 1, 2012 (GB) 1213705.5

(51) Int. Cl. C12Q 1/68; C12N 9/02

(52) U.S. Cl. CPC C12Q 1/6883; C12N 9/0008; C12Q 2600/158; C12Q 2600/118; C12Y 102/01012

(57) ABSTRACT

A method of determining the vascular endothelial function of a subject using GAPDH as a biomarker is presented, whereby the level of GAP-DH mRNA and/or GAPDH protein in a sample obtained from a subject is measured and compared to a reference. A difference in the level of GAPDH mRNA and/or GAPDH protein between that measured for the sample and a reference may be indicative of impaired vascular endothelial function and therefore may indicate the onset of a condition related to impaired vascular endothelial function such as atherosclerosis.

### FIELD OF THE INVENTION

The invention concerns the use of expressed protein and/or messenger RNA for a protein extracted from patient whole blood as a biomarker for vascular endothelial function and integrity.

### BACKGROUND TO THE INVENTION

The endothelium is made up of a layer of cells (endothelial cells) that line the interior surface of blood vessels of the circulatory system (vascular endothelial cells), and lymphatic vessels of the lymphatic system (lymphatic endothelial cells). The main function of the endothelium is to act as a semi-selective barrier controlling the passage of nutrients and white blood cells between the blood vessels and the surrounding tissue in the circulatory system and to allow blood plasma to return from the surrounding tissue to the blood as lymph in the lymphatic system. Other functions include the control of inflammation, angiogenesis (formation of new blood vessels) and vasoconstriction and vasodilation (control of the constriction and dilation of the blood vessels).

Early changes in the normal functioning of the endothelium are key initiating factors in the development and progression of atherosclerosis (also known as arteriosclerotic vascular disease, where the arterial walls thicken due to the build-up of plaques) and they are present well before the presentation of clinical symptoms. To date, a wide range of methods have been proposed to assess endothelial function upon presentation of clinical symptoms, each with its own advantages and limitations.

Examples include skin post occlusion reactive hyperaemia (PORH). PORH in the skin typically involves the use of laser Doppler imaging or laser speckle contrast analysis to determine blood cell mobility in the vicinity of the skin. Laser speckle contrast analysis uses the principle that scattered laser light from objects in the skin tissue layers will form an interference pattern, or speckle pattern, and that moving objects, such as red blood cells, will form a dynamic speckle pattern. Laser Doppler imaging utilises the small change in wavelength of scattered light from a target moving away from or towards the source, such as blood cells, to monitor the speed and density of the said moving blood cells.

The above techniques are limited to assessing blood flow at the skin surface and typically are only able to monitor blood flow over a relatively small area of the skin, of the order of 10 cm², for example. In addition, the above techniques require the availability of specialised equipment and trained operators and are therefore generally restricted to use in specialised medical facilities.

Another technique for measuring vascular function is brachial artery flow-mediated dilation (FMD), whereby the diameter of a section of the brachial artery is measured using ultra-sound (typically) before and after an ischaemia is induced in the patient's forearm.

Therefore, it would be advantageous to provide a method of detecting the presence of impaired vascular endothelial function related to conditions such as atherosclerosis before clinical symptoms are presented, and that does not require expensive and/or bulky specialist equipment.

Accordingly, one of the aims of the present invention is to provide an inexpensive and straightforward method of determining the impairment of vascular endothelial function before clinical symptoms are present.

Once atherosclerosis has been diagnosed, it is necessary to monitor the condition to ensure that the proscribed course of treatment successfully reverses the condition or at least limits progression. The methods known in the art discussed above require specialist equipment and the supervision of a specialist to perform, making them relatively expensive to perform with the frequency that may be required to monitor treatment.

Therefore, a further aim of the invention is to provide a cost effective method of monitoring a regimen of treatment of atherosclerosis.

### SUMMARY OF THE INVENTION

According to a first aspect of the invention there is presented a method of determining vascular endothelial function in a subject, comprising the steps of determining the level of GAPDH mRNA and/or GAPDH protein in a sample obtained from a subject; and comparing the level of GAPDH mRNA and/or GAPDH protein in the obtained sample to that of a reference, wherein a difference between the level of GAPDH mRNA and/or GAPDH protein in the said sample when compared to that of the reference is indicative of a change of vascular endothelial function in the said subject.

The sample obtained from the subject is typically a blood sample.

The subject is typically a human (e.g. a human patient).

The vascular endothelial function that is determined may be the integrity of the vascular endothelium. The vascular endothelial function may be indicative of whether there is dysregulation of normal blood vessel function or a build-up of fatty deposits on the vascular endothelium that may lead to conditions such as atherosclerosis. A numerical value associated with vascular endothelial function may be determined.

By “GAPDH” we refer to glyceraldehyde-3-phosphate dehydrogenase, a fragment or variant thereof. GAPDH is a highly expressed multifunctional protein with diverse physiological functions and activities involved in glycolysis, transcriptional and post-transcriptional gene regulation, vesicular transport, receptor mediated cell signalling, chromatin structure and the maintenance of DNA integrity.

Methods of determining vascular function known in the art including post occlusion reactive hyperaemia and brachial artery flow-mediated dilation (FMD), typically require specialist equipment and image processing. In addition, a specialist is typically required to carry out these procedures and to analyse the data that results from them.

Accordingly, the provision of a method for determining vascular function impairment using a biomarker found naturally in the blood of the patient allows the changes in vascular endothelial function, indicated by a change in the level of GAPDH mRNA and/or GAPDH protein in the blood, for example, to be detected before clinical symptoms are presented, thereby allowing the vascular impairment to be detected and therefore treated at an early stage, thereby reducing risk to the patient.

In addition, the provision of an in vitro method, e.g. that uses a sample taken from the patient by their local medical practitioner at their local surgery or medical centre, which can then be processed to measure at least one property of the biomarker GAPDH mRNA and/or GAPDH protein, is more convenient for the patient and reduces the costs associated with the process.

The reference may be a standard level of GAPDH mRNA and/or GAPDH protein indicative of a healthy vascular endothelial function in a healthy subject. Accordingly, the invention may extend to a method of determining vascular endothelial function in a subject, comprising the steps of determining the level of GAPDH mRNA and/or GAPDH protein in a sample obtained from a subject; and comparing the level of GAPDH mRNA and/or GAPDH protein in the sample to that for a healthy subject,
wherein, a difference between the level of GAPDH mRNA and/or GAPDH protein in the said sample when compared to the level of GAPDH mRNA and/or GAPDH protein of a healthy subject is indicative of a deterioration of vascular endothelial function in the said subject.

The reference against which the obtained sample is compared may be given as a single value above or below which the vascular endothelial function of the subject is determined to be at least partially impaired. Accordingly, the single value may correspond to a threshold value. For example, if the measured level of GAPDH mRNA in the blood of a subject is above or below the threshold value, the vascular endothelial function may be determined to be impaired.

The reference against which the obtained sample is compared may be given as a range of values between which vascular endothelial function is determined to be normal in a healthy subject, and outside of said range vascular endothelial function is determined to be impaired. For example, it may be that above a given range of values for the level of GAPDH mRNA present in the blood of a subject and/or below a given range of values for the level of GAPDH mRNA present in the blood of a subject, vascular endothelial function may be determined to be impaired.

The reference may be a determined level of GAPDH mRNA and/or GAPDH protein in one or more samples obtained from the subject at an earlier date. The reference may be a determined level of GAPDH mRNA and/or GAPDH protein in a sample obtained from the subject prior to the subject undergoing a treatment regimen for a condition related to impaired vascular endothelial function such as atherosclerosis, for example.

The level of GAPDH mRNA in the blood may be measured by using nucleic acid amplification, for example, the reverse transcription polymerase chain reaction (RT-PCR).

Alternatively, the level of GAPDH protein in the blood may be measured directly using standard techniques known in the art, including antibody capture assays such as surface plasmon resonance techniques and enzyme-linked immunoassays, for example. The level of GAPDH protein in the blood may be determined indirectly by measuring an activity of GAPDH. For example, the rate of NAD (Nicotinamide adenine dinucleotide) reduction may be used as a measure of GAPDH activity (as described in a protocol “Glyceraldehyde-3-Phosphate Dehydrogenase Assay” by Worthington Biochemical Corporation of Lakewood N.J., USA).

The method of determining vascular endothelial function in a subject may therefore comprise the steps of; (a) obtaining a sample from a subject; (b) determining the level of GAPDH mRNA and/or GAPDH protein in the obtained sample; and (c) comparing the level of GAPDH mRNA and/or GAPDH protein in the obtained sample to a reference, wherein, a difference between the level of GAPDH mRNA and/or GAPDH protein in the said sample when compared to that of the reference is indicative of a deterioration of vascular endothelial function in the said subject.

The invention extends in a second aspect to a method of determining the efficacy of a treatment regimen for a condition related to impaired vascular endothelial function for a subject comprising the steps of: determining the level of GAPDH mRNA and/or GAPDH protein in one or more samples obtained from a subject before treating the subject for the condition related to impaired vascular endothelial function with a treatment regimen, and in one or more samples obtained from the subject during or after the treatment regimen; and comparing the level of GAPDH mRNA and/or GAPDH protein in the samples,
wherein a difference between the level of GAPDH mRNA and/or GAPDH protein in the one or more samples obtained during or after the treatment regimen when compared to that of the one or more samples obtained prior to the treatment regimen or a change in the rate of change of the level of GAPDH mRNA and/or GAPDH protein is indicative of the efficacy of the treatment regimen.

Preferably, the obtained samples are blood samples. The subject is typically a human (e.g. a human patient).

Once atherosclerosis has been diagnosed, it is advantageous to monitor the progress of any course of treatment as shown by an improvement in vascular endothelial function. The methods known in the art typically require expensive specialist equipment and skilled supervision and require the patient to visit a hospital rather than their local medical centre, which is costly and time consuming.

The provision of a method of determining the efficacy of a treatment regimen for atherosclerosis that simply requires a small amount of blood from the patient is potentially cheaper and more convenient for the patient. In addition, given the high level of accuracy in the art for the measurement of levels of protein and RNA in a sample, the method provides a more accurate measurement of the efficacy of a treatment regimen on the vascular endothelial function of the patient.

The method may comprise the step of obtaining samples during the treatment regimen. The method may comprise the step of obtaining samples after the treatment regimen. The method may comprise the step of obtaining samples during and after the treatment regimen. For example, a sample may be obtained during the treatment regimen and a sample may be obtained after the treatment regimen.

A reduction in the determined level of GAPDH mRNA and/or GAPDH protein in the one or more samples during or after the treatment regimen when compared to that of the one or more samples prior to the treatment regimen may be indicative of the efficacy of the treatment regimen. By indicative of the efficacy of the treatment regimen we mean indicative that the treatment regimen is having a beneficial effect on vascular endothelial function.

An increase in the determined level of GAPDH mRNA and/or GAPDH protein in the one or more samples during or after the treatment regimen when compared to that of the one or more samples prior to the treatment regimen may be indicative of the efficacy of the treatment regimen.

The one or more samples obtained before the beginning of the treatment regimen may be the reference to which the one or more samples obtained during or after the treatment regimen is compared. For example, where one sample is obtained before a treatment regimen and one sample is obtained during the treatment regimen, the one sample obtained before the treatment regimen may be the reference for the one sample obtained during the treatment regimen.

One or more samples may be obtained from the subject after a treatment regimen. For example, where a treatment regimen is provided over a short period of time, one or more samples may be obtained from the subject after the treatment regimen and compared to the one or more samples obtained before the beginning of the treatment regimen. In this way, a change in the level of GAPDH mRNA and/or GAPDH protein in the one or more samples obtained after the treatment regimen when compared to the one or more sample obtained before the beginning of the treatment regimen may be indicative of the efficacy of the treatment regimen.

More than one sample may be obtained during or after the treatment regimen and a change over time in the rate of change in the level of GAPDH mRNA and/or GAPDH protein in the more than one samples obtained during or after the treatment regimen may be indicative of the efficacy of the treatment regimen. A reduction over time in the rate of change in the level of GAPDH mRNA and/or GAPDH protein in the more than one sample obtained during or after the treatment regimen may be indicative of the efficacy of the treatment regimen. A stabilisation of the level of GAPDH mRNA and/or GAPDH protein over time may be indicative of the efficacy of the treatment regimen.

More than one sample may be obtained prior to a treatment regimen.

The more than one sample obtained prior or during a treatment regimen may comprise at least three samples, at least five samples or at least ten samples. Preferably, the more than one sample obtained prior or during a treatment regimen are obtained over a period of time. For example, the more than one samples may be obtained over the course of a week, a month, or a year.

For example, an efficacious treatment regimen may be indicated by a decrease in the level of GAPDH mRNA in the blood of the subject, or an efficacious treatment regimen may be indicated by an increase in the level of GAPDH protein or activity. In embodiments where the level of GAPDH mRNA and/or GAPDH protein in the sample is changing over time prior to the treatment regimen, an efficacious treatment regimen may be indicated by halting or reducing the rate of change in GAPDH protein and/or GAPDH mRNA level in the blood over a period of time.

In particular, in embodiments where the sample is blood, a decrease in the level of GAPDH mRNA and/or GAPDH protein in blood may be indicative of a healthier endothelium and therefore indicative of an efficacious treatment regimen.

The treatment regimen may be a treatment to lower the cholesterol of the subject using a course of drugs, such as statins, for example. The treatment regimen may be a treatment to lower the blood pressure of the subject using a course of drugs such as angiotensin-converting enzyme (ACE) inhibitors, calcium channel blockers or thiazide diuretics, for example. The treatment regimen may comprise the subject taking a drug for a length of time, such as once a day for 3 weeks, for example.

The method of determining the efficacy of a treatment regimen for a condition related to impaired vascular endothelial function for a subject may therefore comprise the steps of (a) obtaining one or more samples from the subject before beginning a treatment regimen; (b) treating the subject for the condition related to impaired vascular endothelial function with a treatment regimen; (c) obtaining one or more samples from the subject during or after the treatment regimen; (d) determining the level of GAPDH mRNA and/or GAPDH protein in each sample; and (e) comparing the level of GAPDH mRNA and/or GAPDH protein in the one or more samples, wherein a difference between the level of GAPDH mRNA and/or GAPDH protein in the one or more samples during or after the treatment regimen when compared to that of the one or more samples prior to the treatment regimen or a change in the rate of change of the level of GAPDH mRNA and/or GAPDH protein is indicative of the efficacy of the treatment regimen.

The invention extends in a third aspect to a vascular endothelial function biomarker comprising;—
(a) GAPDH mRNA or a fragment or variant thereof; or
(b) GAPDH protein or a fragment or variant thereof.

Preferably, the vascular endothelial function biomarker is obtained from the blood of a subject. In particular, the vascular endothelial function biomarker may be extracted from the red blood cells within the blood of a subject.

Methods of determining vascular endothelial function known in the art require expensive, specialised equipment. Highly trained users are required to operate this equipment and to process the data into a form that is indicative of vascular endothelial function. In addition, the methods known in the art require procedures to be carried out on the body of the patient.

The provision of a biomarker for vascular endothelial function found naturally within the blood of a patient allows a simple blood sample to be taken from the patient that can then be analysed using standard in vitro techniques in the field of molecular biology to determine the vascular endothelial function of the patient. Therefore, the provided biomarker allows the vascular endothelial function of a patient to be determined at lower cost and with lower patient involvement, thereby being more convenient for the patient.

Optional features described above in relation to any one of the three aspects of the invention are optional features of any of the aspects of the invention.

### DESCRIPTION OF THE DRAWINGS

An example embodiment of the present invention will now be illustrated with reference to the following Figures in which:

FIG. 1 is a chart of the method according to an embodiment of the invention;

FIG. 2 shows the progress curves for the real time RT-PCR amplification of GAPDH complimentary DNA and the determined threshold level (dashed line);

FIG. 3 is a plot of GAPDH threshold cycles (CO versus baseline skin perfusion;

FIG. 4 is a plot of GAPDH threshold cycles (CO versus post occlusion reactive hyperaemia (PORH);

FIG. 5 is a plot of GAPDH threshold cycles (CO versus peak post occlusion hyperaemia (PORH); and

FIG. 6 is a plot of GAPDH threshold cycles (CO versus brachial artery flow-mediated dilation (FMD) percentage increase.

### DETAILED DESCRIPTION OF AN EXAMPLE EMBODIMENT

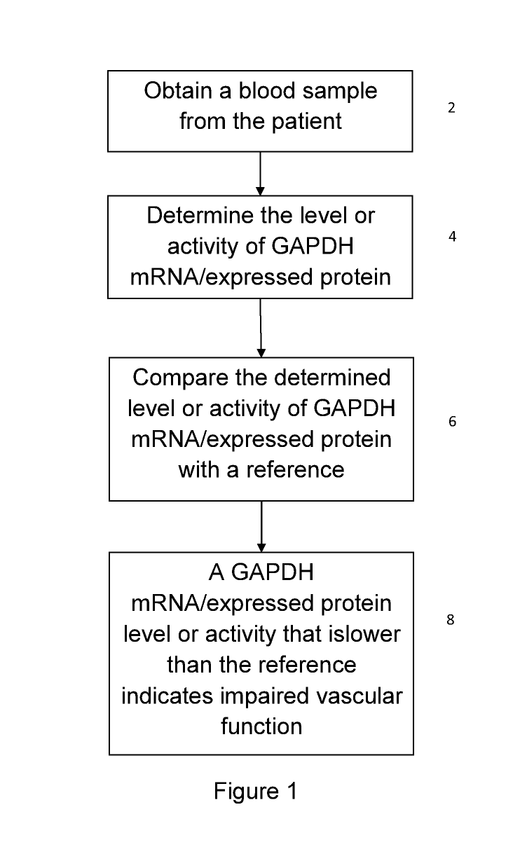

With reference to FIG. 1, in an example application of the invention, a blood sample is taken from a human patient 2, and the blood sample is then analysed 4 in vitro using known methods to determine the level of GAPDH mRNA present (see below). The determined level of GAPDH mRNA present in the blood sample is then compared to a reference GAPDH mRNA level 6, corresponding to that found in a healthy human subject with normal vascular function. If the GAPDH mRNA level is significantly lower than the reference, the human patient is likely to suffer from a lowering or impairment of vascular function 8, which may lead to or be caused by atherosclerosis.

Determining the Relationship Between the Level of GAPDH Protein and mRNA in the Blood and Vascular Function.

The relationship between the level of GAPDH mRNA in the blood of a subject and the vascular function of the subject was determined by correlating the level of GADPH mRNA in the blood and the results of vascular response tests. In particular, the vascular response tests investigated were baseline skin perfusion, post occlusion reactive hyperaemia (PORH) and brachial artery flow-mediated dilation (FMD).

Seventy-five young healthy volunteers (41 males, 34 females) were recruited for the study. None of the subjects were smokers, used any medication or had a history of any symptomatic vascular disease(s). Subject characteristics are shown in Table 1 below.

TABLE 1
Subject Characteristics. Data are presented mean ± SEM.

| | |
|---|---|
| Age (year) | 22.1 ± 0.3 |
| Sex (male/female) | 41/34 |
| Weight (Kg) | 65.1 ± 1.1 |
| Height (m) | 1.70 ± 0.01 |
| Body mass index (kg/m²) | 22.4 ± 0.3 |
| Heart rate (beats/min) | 63.2 ± 1.0 |
| Systolic blood pressure (mmHg) | 115.0 ± 1.2 |
| Diastolic blood pressure (mmHg) | 68.5 ± 0.9 |

The study was approved by the Tayside Committee on Medical Research Ethics and written informed consent was obtained from each subject before participation in the study. All subjects attended for one single visit lasting up to 3 hours during which a blood sample was taken and vascular function tests performed. Vascular assessments were conducted in a blood flow laboratory at a temperature of 23° C. after 10 minutes of acclimatization. Subjects were asked to refrain from food and drink for at least 2 hours beforehand and also to refrain from physical activity for one day before their visit.

Real Time Reverse Transcription-Polymerase Chain Reaction (RT-PCR)

Five millilitres of venous whole blood was collected from a vein in the upper arm of each subject into a heparinised vacutainer. The vacutainer was placed in a sealed transport plastic bag containing ice and sent immediately to the laboratory for determination of levels of GAPDH mRNA by real-time RT-PCR.

Total ribonucleic acid (RNA) was extracted from the human whole blood using TRIZOL reagent (Invitrogen, Paisley, UK) according to manufacturer's recommendations.

The extracted RNA was further purified with RNeasy Mini Kit (Qiangen, Crawley, UK) according to the manufacturer's instruction. The specific primers (shown in Table 2 below) for human GAPDH were designed using Beacon Designer 3.0 software (Premier Biosoft, California, USA).

TABLE 2
Primers for human GAPDH

| | Primer Sequence |
|---|---|
| SEQ.ID.NO. 1 | 5′-GTCTTCACCACCATGGAGAA-3′ |
| SEQ.ID.NO. 2 | 5′-TTCACCACCTTCTTGATGTCA-3′ |

The reverse transcriptase (RT) reaction was carried out with ImProm-II Reverse Transcriptase (Promega, Southampton, UK). A final volume of 20 μl of RT reaction containing 4 μl of 5× buffer, 3 mM MgCl₂, 20 units of RNasin® Ribonuclease inhibitor, 1 unit of ImProm-II reverse transcriptase, 0.5 mM each of deoxyadenosine triphosphate (dATP), deoxycytidine triphosphate (dCTP), deoxyguanosine triphosphate (dGTP), and thymidine triphosphate (dTTP), 0.5 μg of oligo(dT), and 1 μg of RNA was incubated at 42° C. for 1h, then inactivated at 70° C. for 15 minutes. The resulting complementary deoxyribonucleic acid (cDNA) was used as a template for real-time RT-PCR. The SYBR Green I system was used for the RT-PCR test. 25 μl of reaction mixture was used for each sample and contained: 12.5 μl of iQTM SYBR® Green Supermix (2×), 7.5 nM of each primer, 9 μl of double distilled water (ddH₂O), and 2 μl of cDNA. SYBR Green I binds preferentially to double stranded DNA, and the combined DNA/SYBR Green I moiety absorbs blue light (λₘₐₓ=497 mm) and emits green light (λₘₐₓ=520 mm). Therefore, the observed fluorescence is a measure of the amount of double stranded DNA present in the sample and, as any DNA present in the sample has been created from the original complementary DNA template based on the original mRNA, accordingly allows the amount of RNA originally present in the sample to be calculated.

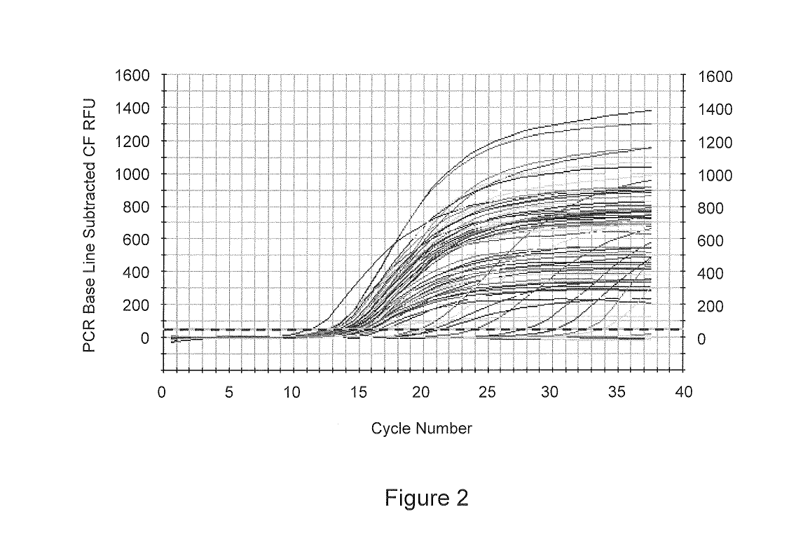

The conditions for thermal cycling were as follows: an initial denaturation at 95° C., 15s of annealing at 56° C., and 30s of extension at 55° C. The real-time PCR was performed in a 96-well plate in the iCycler iQTM MultiColor Real-time Detection System (Bio-Rad, Hercules, Calif., USA). After each cycle, data were collected and shown graphically using the iCycler iQTM Real-time Detection System Software (version 3.0A, Bio-Rad, Hercules, Calif., USA) (see FIG. 2).

Threshold cycle values (or Ct, the PCR cycle number at which the threshold fluorescence, depicted by the dashed line in FIG. 2, is exceeded), PCR efficiency (examined by serially diluting the template cDNA and performing PCR under these conditions) and PCR specificity (determined by melting curve analysis) were determined using the iCycler iQTM Real-time Detection System Software (Bio-Rad, Hercules, Calif., USA).

All experiments were carried out in the presence of blank and positive controls (skeletal muscle).

Assessment of Vascular Function—Baseline Skin Perfusion and Post Occlusion Reactive Hyperaemia (PORH)

Post occlusion reactive hyperaemia (PORH) was tested in 56 subjects (30 males, 26 females). Vascular function was assessed with the subjects lying supine on a bed. The forearm was rested at heart level and the skin microcirculation was measured at the volar aspect using a full field laser perfusion imager (moorFLPI, Moor Instruments Ltd., Axminster, UK). A low-power laser beam was directed by the imager onto the skin surface of the forearm. Superficial microvascular perfusion was measured continuously from five individual regions of interest over an area of approximately 30 cm².

Data collected from the five regions of interest were averaged to provide an overall response in arbitrary perfusion units (PU). A blood pressure cuff was placed over the upper arm and a baseline measurement of skin perfusion was obtained for two minutes. The cuff was then inflated to a suprasystolic pressure (200 mmHg), thus, occluding any blood perfusion distal of the cuff for five minutes, inducing ischaemia distal of the cuff. The cuff was then deflated, immediately resulting in an increase of blood through the skin microcirculation, termed post occlusion reactive hyperaemia (PORH). The peak perfusion post occlusion was measured and in addition, the average perfusion over two minutes following the release of the cuff was determined.

Assessment of Macrovascular Function—Brachial Artery Flow-Mediated Dilatation (FMD)

Brachial artery flow-mediated dilatation (FMD) was tested on 37 subjects (20 males, 17 females). Vascular function was assessed with the subjects lying supine on a bed and according to standard guidelines. The arm was rested at heart level and images of the brachial artery was measured above the antecubital fossa in the longitudinal plane at the volar aspect using high-resolution ultrasound imaging (Acuson Sequoia 512, Siemens Medical Solutions, Berkshire, UK). The ultrasound probe was clamped in place to ensure that a stable image of the brachial artery was obtained throughout the study.

The ischaemic stimulus was produced by placing a blood pressure cuff above the antecubital fossa and inflating to a suprasystolic pressure of around 200 mmHg for five minutes. After the cuff was released, a transient increase in blood flow through the brachial artery was produced by reactive hyperaemia which resulted in an increase in shear stress and dilatation of the brachial artery. 2D images of the brachial artery were acquired for one minute at baseline and for two minutes post cuff release. FMD was calculated as the maximum percentage change in diameter post reactive hypearemia relative to the baseline diameter.

Statistical Analysis

The relationship between levels of GAPDH mRNA (as defined by threshold cycles) in blood and the results of the vascular endothelial tests was assessed using Pearson's correlation coefficient, r, where a value of r=1 corresponds to a perfect positive correlation, r=0 corresponds to no correlation, and r=−1 corresponds to a perfect negative correlation.

Group differences were analyzed using unpaired sample T-tests. Statistical analyses were carried out using SPSS for Windows version 14.0 (SPSS Inc.). Data are presented as mean±SEM and a probability value P<0.05 was considered statistically significant.

Results

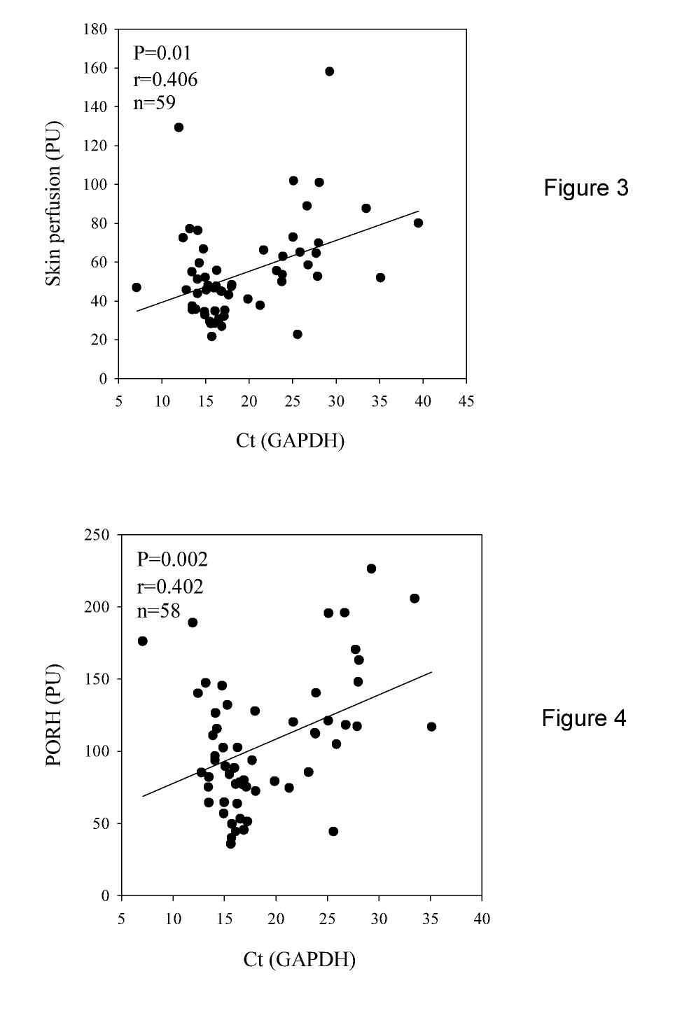

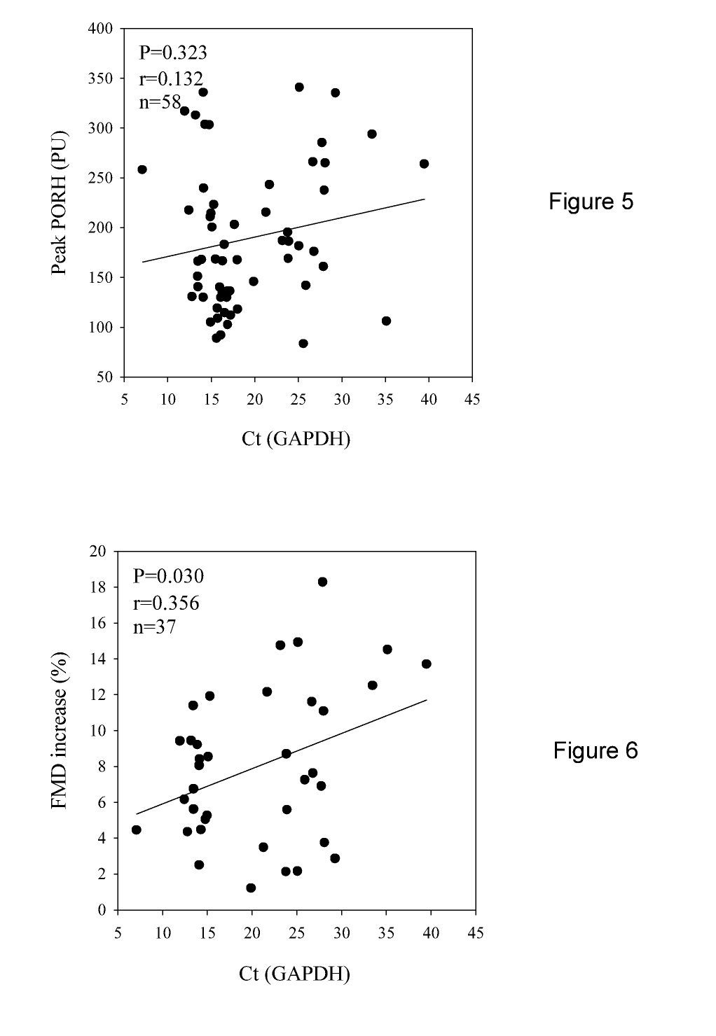

Out of 75 subjects, GAPDH mRNA was detected in the whole blood of 59 subjects (n=59). The average threshold cycle (Cₜ) value for GAPDH was 19.28±0.64. With reference to FIGS. 3, 4 and 6, positive correlations between vascular responses and blood GAPDH mRNA levels were observed. Baseline skin perfusion, the two minute recovery PORH and FMD exhibited significant positive correlations with Cₜ values (baseline skin perfusion: r=0.406, P=0.001, n=59; PORH: r=0.402, P=0.002, n=58; FMD: r=0.356, P=0.030, n=37). A high Ct value equates to lower levels of GAPDH mRNA (i.e. it has taken longer for sufficient double stranded DNA to be produced to which SYBR Green I may bind and fluoresce to exceed the fluorescence threshold), thus a positive correlation means that higher values of basal skin perfusion, two minute recovery PORH and FMD are associated with a lower expression of GAPDH mRNA in the blood. In contrast, there was no significant correlation between Ct values for GAPDH mRNA and peak PORH (r=0.132, P=0.323, n=58).

Discussion

The inventors have determined that a relationship exists between determined vascular functional and the measured GAPDH mRNA level in whole blood in humans. For example, basal perfusion, recovery PORH and FMD, all parameters of vascular function that are known to be dependent on the integrity of the vascular endothelium, have been shown to have a positive correlation to Ct values for GAPDH mRNA levels within the blood of the subject.

In contrast, peak PORH, a parameter that is predominantly independent from the endothelium and can be largely accounted for by the myogenic mechanism, shows little correlation to GAPDH mRNA blood levels. Therefore, blood GAPDH mRNA level correlates with measures of vascular endothelial function.

Given that the level of mRNA in a cell is directly related to the level of expressed protein for which that mRNA is related, it is reasonable to expect the level of GAPDH expressed protein to also be correlated with measures of vascular endothelial function.

The relationship between measured levels of GAPDH mRNA (and therefore potentially measured levels of GAPDH protein), and the integrity of the vascular endothelium may arise from the vascular endothelium's production of nitrogen monoxide (also known as nitric oxide, NO), an important regulator of vascular tone. NO has been reported to either inhibit or activate GAPDH in different cell types. NO produced by the vascular endothelium is known to target blood cells and therefore the inventors hypothesize that NO produced by the vascular endothelium is likely to regulate the level and/or activity of GAPDH in blood cells, thereby explaining the demonstrated relationship between the vascular endothelial function tests and the measured levels of mRNA.

Further variations and modifications may be made within the scope of the invention herein disclosed.

1. A method of determining vascular endothelial function in a subject, comprising: determining the level of GAPDH mRNA and/or GAPDH protein in a sample obtained from a subject; and comparing the level of GAPDH mRNA and/or GAPDH protein in the obtained sample to a reference, wherein a difference between the level of GAPDH mRNA and/or GAPDH protein in the said sample when compared to that of the reference is indicative of a deterioration of vascular endothelial function in the said subject.

2. A method according to claim 1, wherein the reference is a standard level of GAPDH mRNA and/or GAPDH protein indicative of a healthy vascular endothelial function in a healthy subject.

3. A method according to claim 1, wherein the reference is a range of levels of GAPDH mRNA and/or GAPDH protein indicative of a healthy vascular endothelial function in a healthy subject.

4. A method according to claim 1, wherein the vascular endothelial function that is determined is the integrity of the vascular endothelium.

5. A method according to claim 4, wherein a decrease in the integrity of the vascular endothelium is indicative of atherosclerosis.

6. A method according to claim 1, wherein the sample is a blood sample.

7. A method according to claim 1, wherein the subject is a human.

8. A method according to claim 1, wherein the level of GAPDH mRNA is detected using reverse transcription polymerase chain reaction (RT-PCR).

9. A method according to claim 1, wherein the level of GAPDH protein is determined indirectly by measuring an activity of GAPDH protein present in the sample.

10. A method of determining the efficacy of a treatment regimen for a condition related to impaired vascular endothelial function for a subject comprising: determining the level of GAPDH mRNA and/or GAPDH protein in one or more samples obtained from a subject before treating the subject for the condition related to impaired vascular endothelial function with a treatment regimen, and in one or more samples obtained from the subject during or after the treatment regimen; and comparing the level of GAPDH mRNA and/or GAPDH protein in the samples;
wherein a difference between the level of GAPDH mRNA and/or GAPDH protein in the one or more samples obtained during or after the treatment regimen when compared to that of the one or more samples obtained prior to the treatment regimen or a change in the rate of change of the level of GAPDH mRNA and/or GAPDH protein is indicative of the efficacy of the treatment regimen.

11. A method according to claim 10, wherein a reduction in the determined level of GAPDH mRNA and/or GAPDH protein in the one or more samples during or after the treatment regimen when compared to that of the one or more samples prior to the treatment regimen is indicative of the efficacy of the treatment regimen.

12. A method according to claim 10, wherein an increase in the determined level of GAPDH mRNA and/or GAPDH protein in the one or more samples during or after the treatment regimen when compared to that of the one or more samples prior to the treatment regimen is indicative of the efficacy of the treatment regimen.

13. A method according to claim 10, wherein more than one sample is obtained during or after the treatment regimen and a change over time in the rate of change in the level of GAPDH mRNA and/or GAPDH protein in the more than one samples obtained during or after the treatment regimen is indicative of the efficacy of the treatment regimen.

14. A method according to claim 13, wherein a reduction over time in the rate of change in the level of GAPDH mRNA and/or GAPDH protein in the more than one sample obtained during or after the treatment regimen is indicative of the efficacy of the treatment regimen.

15. A vascular endothelial function biomarker comprising:
(a) GAPDH mRNA or a fragment or variant thereof; or
(b) GAPDH protein or a fragment or variant thereof.
